(19) United States
(12) Patent Application Publication
(10) Pub. No.: US 2025/0332302 A1
(43) Pub. Date: Oct. 30, 2025

(54) Perioperative Cobotic System for Monitoring Bioburden

(71) Applicant: Martin Roche, Fort Lauderdale, FL (US)

(72) Inventor: Martin Roche, Fort Lauderdale, FL (US)

(21) Appl. No.: 19/195,731

(22) Filed: Apr. 30, 2025

Related U.S. Application Data
(60) Provisional application No. 63/640,524, filed on Apr. 30, 2024.

(51) Int. Cl. A61L 2/00; A61L 2/10; A61L 2/24; A61L 2/28

(52) U.S. Cl. CPC A61L 2/0088; A61L 2/10; A61L 2/24; A61L 2/28; A61L 2202/10; A61L 2202/25

(57) ABSTRACT

The invention relates to a perioperative cobotic system designed to monitor, assess, and manage bioburden in real-time within operating room environments during surgical procedures and turnover phases. The system includes a mobile base equipped with a drive mechanism for maneuverability, a camera to capture image data, an actuator for camera movement, and a biosensor for direct bioburden assessment.

### CROSS-REFERENCE TO RELATED APPLICATIONS

This patent application claims the benefit of priority to U.S. Provisional Patent Application No. 63/640,524, filed on Apr. 30, 2024, the entirety of which is incorporated herein by reference.

### TECHNICAL FIELD

The present invention relates generally to cobotic systems, and more specifically to a perioperative cobotic system for monitoring bioburden accumulation in an operating room.

### BACKGROUND

Surgical procedures involve complex interactions between surgical teams and the operative environment to promote the safety of both patient and hospital staff and positive surgical outcomes for the patient. One aspect of these interactions is monitoring and managing contamination and microscopic bioburden that accumulates within the operating room perioperatively. The presence of excessive bioburden in an operating room can lead to increased risk to both patient and staff of surgical site infections, which can lead to morbidity and extended hospital stays.

Traditional methods for managing bioburden in an operating room generally lack the ability to provide continuous monitoring and real-time bioburden levels. Additionally, such methods generally rely on compliance with infection control policies but lack robust mechanisms to clean, decontaminate, monitor and audit operating room activities for compliance. Thus, there exists a need for an improved system to assist in monitoring and managing bioburden in an operating room environment.

### SUMMARY

Embodiments disclosed herein relate to a cobotic system designed for monitoring, assessing, and managing bioburden in operating rooms during surgical procedures and turnover phases. The system comprises a mobile base, a camera, an actuator, and a biosensor, all coordinated by a control system. The mobile base, equipped with a drive mechanism, navigates throughout the operating room, enabling the system to position itself based on the operational requirements of the surgical environment.

The camera, mounted on the mobile base and maneuvered by the actuator, captures real-time image data of the surgical procedure and surrounding area. This image data is processed by the control system to identify areas within the operating room at increased risk of bioburden accumulation. Upon identification, the system's biosensor, which may be mounted at the end of a robotic arm for precise placement, is deployed to assess bioburden levels directly, providing immediate and accurate assessment of the sterility and contamination levels in the identified region.

Embodiments also include modular features such as interchangeable biosensors tailored for different types of bioburden detection, such as microbial, chemical, or particulate matter sensors, enhancing the system's utility across various healthcare settings and requirements.

Furthermore, an integrated display unit visually conveys critical data, such as bioburden levels and specific contamination locations, to the surgical team, assisting in decision-making and guiding cleaning or sterilization procedures necessary to maintain sterility standards. A wireless communication module allows for the transfer of bioburden data to a centralized database, facilitating broader analyses and strategy adjustments in hospital infection control practices.

Additionally, the system's control scheme incorporates machine learning algorithms to enhance predictive capabilities and operational efficiency. By analyzing accumulated data, the system can predict areas at risk of contamination and streamline its monitoring tasks.

Overall, such cobotic systems represent advancements in robotic assistance within healthcare environments, focusing on maintaining clean and safe conditions, potentially reducing procedural complications and enhancing patient and staff safety.

### BRIEF DESCRIPTION OF DRAWINGS

The foregoing and other objects, aspects, features, and advantages of the disclosure will become more apparent and better understood by referring to the following description taken in conjunction with the accompanying drawings, in which:

FIG. 1 is a perspective view of a perioperative cobot;

FIG. 2 is a front view of a perioperative cobot;

FIG. 3 is a front view of a humanoid perioperative cobot;

FIG. 4 is a perspective view of a humanoid perioperative cobot with a wheeled base;

FIG. 5 is a block diagram of a perioperative cobot, in accordance with certain embodiments; and

FIG. 6 is a flow chart of a process for monitoring bioburden associated with perioperative activities in an operating room.

### DETAILED DESCRIPTION

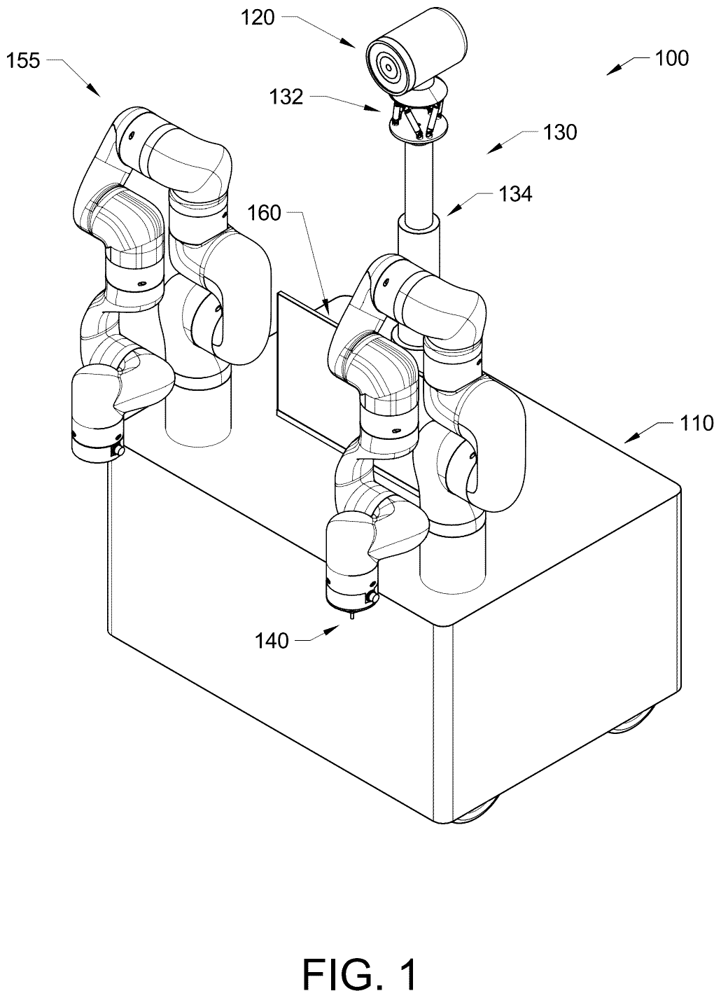

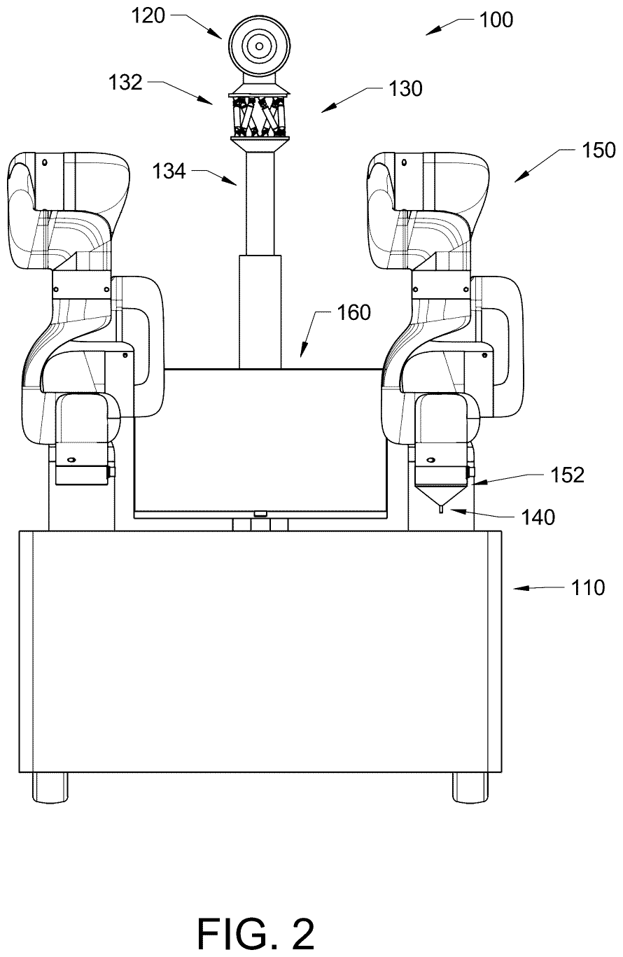

Referring now in detail to the drawings, FIGS. 1 and 2 depict a perioperative cobotic system 100 to monitor and assess bioburden in real-time during surgical procedures and turnover within an operating room environment. As used herein, “turnover” refers to the processes that occur between adjacent surgical procedures, including room setup and equipment preparation prior to a surgical procedure, as well as cleaning and disinfection after a surgical procedure. Moreover, “perioperative” includes, without limitation, surgical procedures and turnover.

The perioperative cobotic system 100 includes a mobile base 110, which serves as the foundational platform for stability, mobility and maneuverability within the operating room. The mobile base 110 is equipped with a drive mechanism that is responsible for the movement and maneuverability of the mobile base 110. In an embodiment the mobile base 110 houses a battery or other power supply to provide the power to the perioperative cobotic system 100; power management circuitry, plugs and other component facilitate battery recharging; and computer hardware included in the control system 170, such as a central processing unit, a hard drive, random access memory, and various other electronic components.

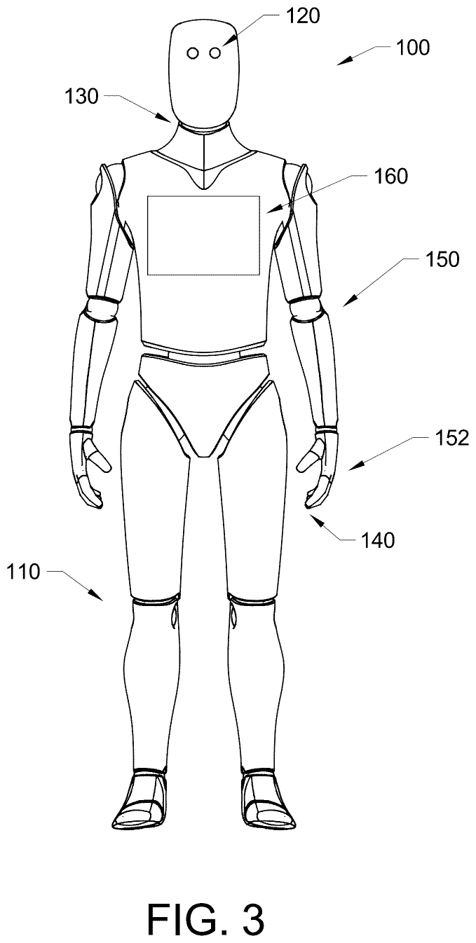

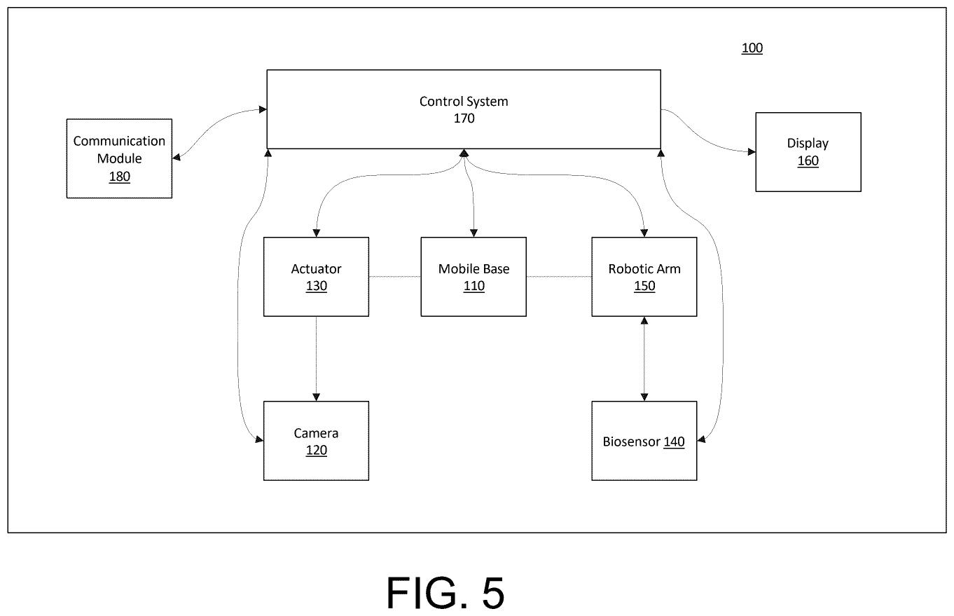

The drive mechanism generates and applies a force to move the mobile base 110 in a particular direction. This is generally achieved through electric motors, hydraulic or pneumatic actuators, or similar mechanisms. The drive mechanism also includes systems that allow the mobile base 110 to vary its speed, navigate turns, and adjust its orientation. As depicted in FIG. 3, in some embodiments the drive mechanism interfaces with a bipedal or quadrupedal mobile base 100 design that mimics the gait of humans or quadrupeds, respectively. As depicted in FIGS. 1, 2 and 4, in other embodiments the drive mechanism employs a differential drive system with conventional wheels, or a holonomic drive system with omni-directional wheels. The drive mechanism is regulated by the control system 170, as shown in FIG. 5, to dynamically position the perioperative cobotic system 100 within the operating room. The mobile base 110 utilizes on board sensors to allow it to move about the room in a controlled and prescriptive manner while avoiding objects, people and the sterile field, which could lead to contamination.

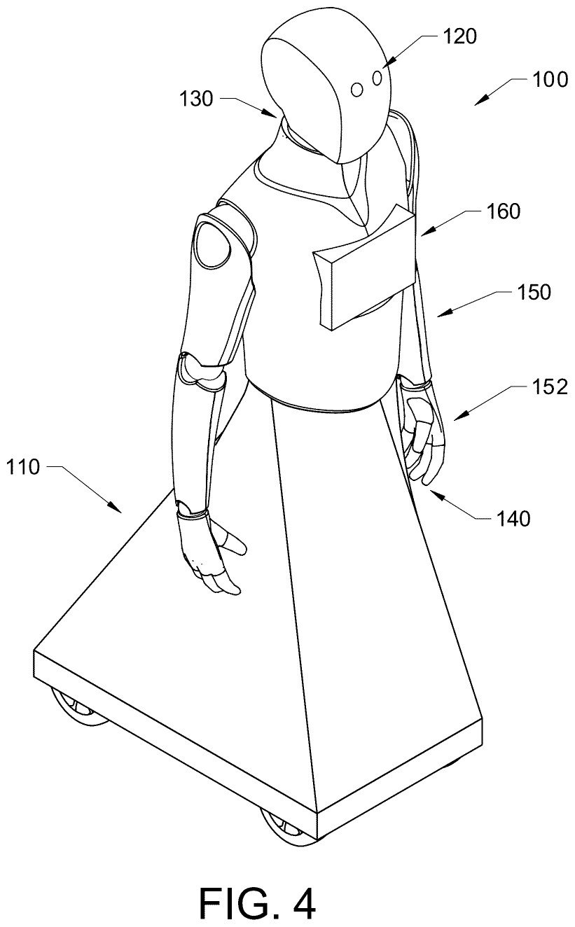

A camera 120 is coupled to the mobile base 110. The camera 120 collects image data relating to a surgical procedure and turnover in the context of an operating room environment. In various embodiments image data includes, for example, sequences of bitmap images, sequences of point clouds, compressed video segments, or other representations of the surrounding environment generated at wavelengths above, below or within the optical spectrum. In other embodiments camera 120 includes stereoscopic configurations and multiple imaging modalities, such as an optical camera capturing video in the optical band together with a LiDAR system capturing point cloud data at a wavelength in the near-infrared band. In the embodiment illustrated in FIGS. 3 and 4, the camera 120 is situated within an eye socket of the humanoid frame. In an embodiment, the camera 120 has several different sensors to enable it to evaluate known morphology of instruments and assets within the operating room such as operative beds, lights and monitors. The optic sensors can be Amino fluorescent and other wavelengths that enable the detection of damaged instruments and or instruments or surfaces contaminated with bioburden directly or when reacting to a surface agent that is sprayed on, or otherwise applied to, the object.

Image data collected by the camera 120 is transmitted to the control system 170. In some embodiments the camera 120 provides raw image data to the control system 170. In other embodiments the camera 120 provides compressed, encoded or otherwise preprocessed image data. The control system 170 transmits commands to the camera 120 relating to the acquisition of the image data, such as, by way of example, when to begin and end data acquisition, data acquisition rates, and focal length adjustments.

To provide the camera 120 with an unobstructed view of an area of interest, as determined by the control system 170 or a user, an actuator 130 is provided between the camera 120 and the mobile base 110. The actuator 130 may, for example, take the form of a robotic joint modeled after the human neck and capable of flexion, extension, left and right lateral rotation, and left and right lateral flexion. The actuator 130 may also provide an additional translational degree of freedom to facilitate adjustments to the height of the camera 120 relative to the mobile base 110.

The actuator 130 is regulated by the control system 170 to maneuver the camera 120 relative to the mobile base 110. Accordingly, the control system 170 directs the position of the mobile base 110 within the operating room environment by regulating the drive system, and the position and orientation of the camera 120 relative to the mobile base 110 by regulating the actuator 130. In some embodiments this configuration results in redundant degrees of freedom of the camera 120 relative to the operating room environment to enable the control system 170 to keep the surgical procedure within the field of view of the camera 120 concurrent with ongoing movements and changes within the operating room, obstacle avoidance, avoiding impinging on the sterile field and various other considerations. The actuator 130 includes telescoping features that enable it to enter the sterile field without contaminating or obstructing the surgical team to enable visualization of the instrument trays, robotic systems, and the live surgical field.

The perioperative cobotic system 100 includes a biosensor 140, which is also coupled to the mobile base 110. In an embodiment, a robotic arm 150 is coupled to the mobile base 110 and the biosensor 140 is coupled to a distal end 155 of the robotic arm 150 to facilitate positioning the biosensor 140 proximate to a local region of interest. In this context, “proximate” means the biosensor 140 is sufficiently close to the local region of interest to obtain a useful bioburden measurement and may, depending on the form of biosensor, include bringing the biosensor 140 into contact with the local region of interest.

In an embodiment the robotic arm 150 may, for example, be an articulated robotic arm with four to seven degrees of freedom and a sufficient working envelope to position the biosensor 140 proximate to objects and structures anticipated to contain local regions of interest, such as floors, walls, tables, carts, and so forth. In another embodiment the robotic arm 150 is a simplified configuration with one translational and one rotational degree of freedom. In other embodiments the robotic arm 150 mimics a human arm.

The biosensor 140 can, by way of example, be a microbial detection sensor, a chemical detection sensor, a particulate matter sensor, or another biosensor modality, or combination of biosensor modalities, appropriate for the bioburden of interest in a given setting. The biosensor 140 is deployed, using the robotic arm 150, to directly assess the bioburden level in local regions of the operating room environment identified by the control system 170 as having an increased risk of bioburden accumulation or predetermined and specified to the control system 170 by a user. The direct measurement of bioburden in a local region with the biosensor 140 provides quantitative data on the sterility of the environment, facilitating real-time, informed bioburden management.

In another embodiment, the perioperative cobotic system 100 includes a plurality of mechanically interchangeable biosensors with distinct modalities, and biosensor 140 is a modality, or combination of modalities, selected by the control system 170.

The control system 170 coordinates the activities of the mobile base 110, actuator 130, camera 120, and biosensor 140. It processes image data from the camera 120 to identify local regions of the operating room at increased risk of bioburden accumulation and to safely navigate the operating room. Upon identifying a local region having an increased risk of bioburden accumulation, the control system 170 directs relevant components of the perioperative cobotic system 100 to position the biosensor 140 proximate to this region to assess the bioburden. Furthermore, the control system 170 is capable of generating, storing, and displaying bioburden data, facilitating the creation of a dynamic bioburden map of the operating room. In an embodiment, control system 170 also correlates bioburden levels with identified activities associated with the surgical procedure to provide insight into operational factors contributing to increased bioburden in the operating room. In an embodiment the control system 170 directs the collection of multiple samples taken from various surfaces after cleaning a room to confirm and validate decontamination of the room.

A display 160 in communication with the control system 170 provides a visual indication of the location and measured level of bioburden detected. In an embodiment, the display 160 also shows estimated bioburden levels associated with local regions where the bioburden has not been directly measured with biosensor 140 based on a combination of image data and direct bioburden assessments in other local regions. When measured or estimated bioburden in a local region is not in compliance with applicable infection control policies, display 160 informs the surgical staff of the non-compliance in real-time to inform decisions regarding immediate remedial actions.

The perioperative cobotic system 100 further includes a communication module 180 that facilitates the wireless or wired transfer of bioburden measurement data and raw collected data to a centralized facility database. A centralized facility database will generally have access to significantly more data storage and computation power than those resources available on-board the perioperative cobotic system 100. In this manner, a more robust and resource-intensive analysis of the data collected by the perioperative cobotic system 100 can be performed after the surgical procedure to identify improvements to the on-board, real-time analysis of the perioperative cobotic system 100.

In an embodiment, the data utilized to identify local regions of the operating room having increased risk of bioburden accumulation are paired with respective biosensor 140 measurements to generate training data sets in which the available utilized data is transformed to an input vector or tensor, and the biosensor 140 measurements provide ground truth labels. As the corpus of labeled training data grows, it can be sued to refine and improve predictive models for identifying, prior to direct measurement with biosensor 140, local regions with increased risk of bioburden accumulation.

FIGS. 1 and 2 depict an embodiment of a cobotic system 100 with articulated robotic arms 150 mounted on a wheeled mobile base 110. FIG. 3 depicts an embodiment of a cobotic system 100 with a bipedal humanoid form. FIG. 4 depicts an embodiment of a cobotic system 100 with a humanoid torso, head and arms 150 on a wheeled mobile base 110.

Now turning to FIG. 5, a block diagram of the perioperative cobotic system 100, in accordance with certain embodiments, is presented to illustrate the interconnectivity and arrangement of certain components of the perioperative cobotic system 100. The control system 170 coordinates and regulates the activities of various system components, including the mobile base 110, the camera 120, the actuator 130, the biosensor 140, the display 160, and the communication module 180.

The control system 170 analyzes inputs from the camera 120 to identify local regions within the operating room having an increased risk of bioburden accumulation. In an embodiment this analysis initially utilizes a predictive algorithmic or heuristic methodology incorporating the expertise of surgeons or other domain experts, while collecting data sets that can be used to train a machine learning model. According to this embodiment, after a sufficient amount of training data has been collected to provide reliable predictions, the analysis of image data to identify local regions with an increased risk of bioburden accumulation is migrated to a machine learning model, which is continuously improved with the collection of additional training sets.

The mobile base 110, powered and directed by the drive mechanism, provides the foundational mobility required for the system to traverse the operating room environment. In an embodiment the control system 170 also utilizes the image data from the camera 120 for autonomous movement of the perioperative cobotic system 100 within the operating room. In another embodiment a separate imaging system is configured as a dedicated guidance system to facilitate autonomous movement of the mobile base 110. During autonomous movement, the drive mechanism is carefully regulated by the control system 170 to avoid surgical staff and obstacles, to avoiding impinging upon the sterile field, and to generally effect safe operation as moves to perform bioburden assessments in identified regions of interest.

Camera 120, positioned by the actuator 130, captures image data of the operating room, the surgical procedure, and various other perioperative activities. The actuator 130 adjusts the orientation and position of the camera 120 to maintain the surgical procedure or other regions of interest within the field of view of the camera 120, as directed by the control system 170.

The biosensor 140, which can be a singular sensor or selected from a suite of interchangeable sensors, is deployed to points of interest identified through image analysis. Coupled to the distal end 155 of the robotic arm 150, the biosensor 140 is positioned proximate to the targeted surfaces, or air spaces in some circumstances, to directly measure bioburden levels.

Data obtained from the biosensor 140, together with image data, is used by the control system 170 to generate real-time reports of bioburden levels and to create a bioburden map of the operating room. This map is dynamically updated and can be displayed on the display 160, providing actionable information to surgical staff regarding bioburden accumulation and regions within the operating room that may no longer be in compliance with infection control policies.

The control system 170 also correlates identified bioburden levels with specific surgical activities, assigning time stamps to these events, which can provide valuable supplemental data for analysis and identification of bioburden trends over the course of surgical procedures. In an embodiment the control system 170 employs facial recognition and activity classification to identify members of the hospital staff and to classify activities they are performing at any given time. This correlation data, along with raw and processed information from the camera 120 and the biosensor 140, can be transmitted via the communication module 180 to a centralized database for further analysis, model training and record-keeping.

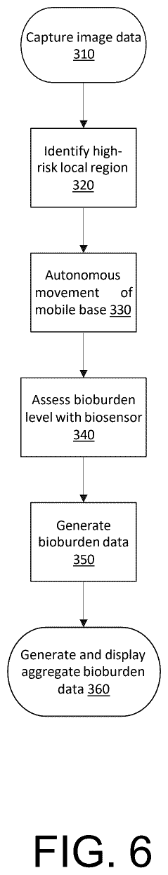

Turning now to FIG. 6, this figure illustrates a flow chart of a process for monitoring bioburden associated with perioperative activities in an operating room using the perioperative cobotic system 100. The process begins at step 310, where the camera 120 is positioned to capture images of the perioperative activities within its field of view. This initiates a monitoring sequence, facilitating the continuous or interval-based collection of visual data pertinent to the operating environment and ongoing procedure.

At step 320, the captured image data is transmitted to the control system 170, where it is analyzed to identify local regions within the operating room that exhibit an increased risk of bioburden accumulation. This processing may utilize a combination of algorithms, heuristics, and machine learning models to accurately discern areas of concern based on the image data.

Following the identification of a high-risk local region, step 330 involves the autonomous movement of the mobile base 110 to maneuver the biosensor 140 into close proximity with the high-risk local region. This autonomous movement takes into account the dynamic nature of the operating room environment, employing obstacle avoidance algorithms and adherence to sterile field protocols to ensure that the perioperative cobotic system 100 does not impinge upon the sterile field or interrupt surgical staff.

At step 340, the biosensor 140 is deployed to directly assess the bioburden level within the local region. The biosensor modality, or modalities, utilized, whether it is, for example, a microbial detection sensor, a chemical detection sensor, a particulate matter sensor, or a combination thereof, may be determined based on the location and nature of the identified risk or other classification of the bioburden, leveraging the system's capacity for interchangeable biosensors to apply the most appropriate assessment technique. In an embodiment, steps 330 and 340 are repeated to assess bioburden at multiple high-risk local regions.

Upon completion of the bioburden assessment, step 350 involves the control system 170 generating bioburden data indicative of the measured levels of contamination. This bioburden data informs real-time decision-making, equipping the surgical staff to promptly address potential contamination risks.

In an embodiment, step 350 includes using robotic arms 150 to remove the bioburden using an appropriate disinfectant system including sprays such as bleach ammonia and cloths or scrub brushes to mechanically clean a surface or object with detergent such as glutaraldehyde prior to opening of sterile trays. Another embodiment utilizes different modalities such as, for example, hydrogen peroxide or UV light. The identified bioburden, location and cleaning method used to clean a surface or object are recorded in a centralized database.

The process ends at step 360, where the aggregate bioburden data, including the locations and assessed bioburden levels, is reported and stored. This final step includes the display of the bioburden map on the display 160 and may include the transmission of the bioburden data to a centralized database for record-keeping, analysis, and to inform broader infection control policies and strategies. The described process underscores the capability of the perioperative cobotic system 100 to autonomously monitor, identify, and assess bioburden risks within the operating room, thereby contributing to the maintenance of sterile environments and enhancing the safety of patients and hospital staff during surgical procedures.

Although exemplary embodiments of the present disclosure have been described in detail, those skilled in the art will appreciate that various changes, substitutions and improvements disclosed herein may be made without departing from the spirit and scope of the disclosure in its broadest form. As used herein, the term “include” and similar terms mean inclusion without limitation, and the term “or” is inclusive, meaning and/or.

The description and drawings in the present disclosure should not be read as implying that any particular element, step, function or advantage is an essential element that must be included in the claim scope. The scope of patented subject matter is defined only by the allowed claims.

What is claimed is:

1. A cobotic system for monitoring bioburden associated with a surgical procedure in an operating room comprising:
a mobile base;
a camera for capturing image data related to the surgical procedure within a field of view;
an actuator configured to move the camera relative to the mobile base;
a biosensor coupled to the mobile base; and
a control system in communication with the mobile base, the actuator, the camera, and the biosensor, the control system configured to:
control the actuator to maintain the surgical procedure within the field of view,
identify, based on the image data, a local region of the operating room having an increased risk of bioburden accumulation,
move the mobile base according to the image data to position the biosensor proximate to the local region,
deploy the biosensor to assess a bioburden level in the local region, and
generate bioburden data indicative of the bioburden level in the local region.

2. The system of claim 1 further comprising a display, wherein the control system is further configured to indicate the location and the bioburden level on the display.

3. The system of claim 2, wherein the control system is further configured to:
compile a bioburden map of the operating room including the bioburden data, and
display the bioburden map on the display.

4. The system of claim 1, wherein the biosensor is selected from a group consisting of a microbial detection sensor, a chemical detection sensor, a particulate matter sensor, and a combination of two or more thereof.

5. The system of claim 1, wherein the control system is further configured to correlate the bioburden data with specific phases of the surgical procedure based on timestamps.

6. The system of claim 1, wherein the actuator is a robotic joint capable of flexion, extension, left lateral rotation and right lateral rotation.

7. The system of claim 1 further comprising a wireless communication module for transmitting the bioburden data to a database.

8. The system of claim 1, wherein the control system is further configured to detect and avoid obstacles while moving the mobile base.

9. The system of claim 8, wherein the control system is further configured to avoid impinging upon a sterile field in the operating room while moving the mobile base.

10. A method for monitoring bioburden associated with perioperative activities in an operating room using a cobotic system, the method comprising:
positioning a camera coupled to a mobile base to capture images of the perioperative activities;
processing the images with a control system to identify a local region of the operating room having an increased risk of bioburden accumulation;
moving the mobile base according to the image data to bring a biosensor into proximity with the local region; and
deploying a biosensor to assess a bioburden level in the local region.

11. The method of claim 10, wherein processing the image data includes employing a machine learning algorithm to identify the local region of the operating room having an increased risk of bioburden accumulation.

12. The method of claim 10, further comprising showing the identified local region and an indication of the assessed bioburden level on a display.

13. The method of claim 10, wherein moving the mobile base includes utilizing a guidance system configured to autonomously move the mobile base avoiding obstacles in the operating room.

14. The method of claim 13, wherein the guidance system is further configured to identify a sterile field in the operating room and to avoid impinging on the sterile field.

15. The method of claim 10, further comprising:
classifying the perioperative activities associated with the surgical procedure using a machine learning classification system to generate a plurality of labeled activities; and
identifying a precipitating activity from the plurality of labeled activities that is responsible for the increased risk of bioburden accumulation.

16. The method of claim 10, further comprising:
cleaning the local region with a robotic arm coupled to the mobile base; and
recording details associated with the cleaning in a centralized database.

17. The method of claim 10, further comprising selectively cleaning the local region based on the assessed bioburden level.

18. The method of claim 10, further comprising:
assessing a second bioburden level in a second region of the operating room;
generating a bioburden map based on the assessments of the bioburden levels in the local region and the second region.

19. A cobotic system for use in an operating room during a surgical procedure, the system comprising:
a mobile base equipped with a drive mechanism for navigating the operating room;
a camera for capturing image data related to the surgical procedure within a field of view;
an actuator configured to move the camera to maintain the surgical procedure within the field of view;
a robotic arm coupled to the mobile base;
a biosensor coupled to a distal end of the robotic arm;
a control system operatively connected to the drive mechanism, camera, actuator, robotic arm, and biosensor, the control system configured to:
process the image data from the camera to identify a local region at increased risk of bioburden accumulation based on image analysis,
regulate the drive mechanism and the robotic arm to position the biosensor in proximity to the local region,
communicate with the biosensor to obtain bioburden measurement data from the local region,
a communication interface for transmitting recorded bioburden data and receiving operational commands;
a power source contained within the mobile unit; and
an obstacle detection and avoidance system integrated with the control system to facilitate unimpeded movement of the mobile unit while avoiding interference with sterile zones and surgical staff, wherein the system is designed to autonomously adapt to dynamic conditions within the operating room and execute preventive actions to maintain a controlled bioburden environment.

20. The system of claim 19, wherein the biosensor is selected from a plurality of interchangeable biosensors, and the control system is further configured to:
classify the identified bioburden accumulation, and
select the biosensor from the plurality of interchangeable biosensors based on the classification of the identified bioburden accumulation.
